(19) United States
(12) Patent Application Publication
(10) Pub. No.: US 2015/0068427 A1
(43) Pub. Date: Mar. 12, 2015

(54) Optical Film

(71) Applicant: SK Innovation Co., Ltd., Seoul (KR)

(72) Inventors: Won Yeob Kim, Daejeon (KR); Myoung Lae Kim, Daejeon (KR); Hyuk Jun Kim, Daejeon (KR); Yong Gyun Cho, Daejeon (KR); Min Joung Im, Busan (KR); Yeong Min Jo, Seoul (KR); Seung Eon Lee, Daejeon (KR); Hyo Shin Kwak, Daejeon (KR)

(21) Appl. No.: 14/399,294

(22) PCT Filed: May 7, 2013
(86) PCT No.: PCT/KR2013/003932

§ 371 Date: Nov. 6, 2014

(30) Foreign Application Priority Data
May 7, 2012 (KR) 10-2012-0047880

(51) Int. Cl. C08K 5/07; C07C 69/732; C08K 5/101; C07C 49/84

(52) U.S. Cl. CPC C08K 5/07; C07C 49/84; C07C 69/732; C08K 5/101
USPC 106/170.39; 568/331; 560/57; 560/60; 106/170.48

(57) ABSTRACT

Provided is an optical film used for the optical compensation, in which additives are added to thereby have a good stability at high temperature and high humidity, a higher chromaticity, a higher Haze and a higher moisture resistance, as well as a small thickness direction retardation (Rth).

### TECHNICAL FIELD

The present invention relates to an optical film used for optical compensation, and more particularly, to an optical film having a small thickness direction retardation (Rth).

Furthermore, the present invention relates to an optical structure and a display device, in which the optical film is used.

### BACKGROUND ART

A cellulose acetate film, which has been widely used as an optical film, has been used for a great variety of photographs or optical materials due to a strong strength and fire retardancy. The cellulose acetate film has an optical anisotropy lower than other polymer films and thus has a relatively low retardation. Therefore, such a cellulose acetate film has been used for a polarization plate or the like.

Recently, a demand of a liquid crystal display device which has higher function such as image quality improvement has increased, and a cellulose acetate film for a polarization plate as materials which form the liquid crystal display device has been also required to satisfy the higher function. Particularly, there is demanded that in an In-Plain Switching (IPS) Mode liquid crystal display device, it is determined to provide a low optical anisotropy of the cellulose acetate film (Re: film in-plane retardation, Rth: film thickness direction retardation) by a method for improving a color shift and contrast ratio.

Korean Patent Laid-open Publication No. 2008-0106109 (Patent Document 1) discloses the compound which is allowed to decrease a thickness direction retardation, but mainly discloses addition of a mathacrylic acid-based polymer and a wavelength dispersion regulator. Japanese Patent Laid-open Publication No. 2011-089005 (Patent Document 2) discloses a technology that improves a moisture resistance by adding a citric acid derivative, but has no disclosure of a reduction in a thickness direction retardation.

Therefore, there is a need to develop a cellulose acetate film of which a thickness direction retardation is sufficiently reduced while haze, a heat resistance, and a moisture resistance of the film are improved.

### RELATED ART DOCUMENT

(Patent Document 1) Korean Patent Laid-open Publication No. 2008-0106109

(Patent Document 2) Japanese Patent Laid-open Publication No. 2011-089005

### DISCLOSURE

### Technical Problem

An object of the present invention is to provide an optical film, particularly, an optical film which has a small thickness direction retardation. More specifically, the present invention is to provide an optical compensation film which may be used for an IPS Mode liquid crystal display device and in which color shift and a contrast ratio of the IPS Mode liquid crystal display device are improved.

Furthermore, another object of the present invention is to provide an additive to satisfy such optical features.

Furthermore, another object of the present invention is to provide an optical structure and a display device, in which the optical film is used.

### Technical Solution

In one general aspect, there is provided an optical film including:

at least one selected from compounds represented by the following Chemical Formula 1, an additive.

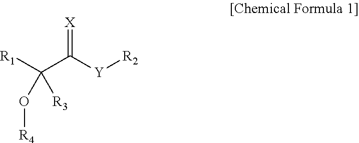

(In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl, R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl, R₁, R₂, R₃ and R₄ are not hydrogen at the same time, and

alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ may be further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.)

In another general aspect, there is provided an optical film composition including: 1 to 20 part(s) by weight of at least one selected from the compounds in the following Chemical Formula 1 with respect to 100 parts by weight of a base resin, as an additive.

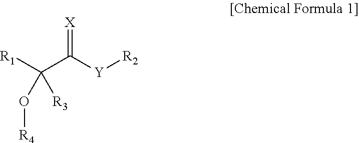

(In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl, R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl, R₁, R₂, R₃ and R₄ are not hydrogen at the same time, and

alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ may be further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.)

In another general aspect, there is provided an optical structure and a display device, in which the optical film is used.

### Advantageous Effects

The optical film according to the present invention can have a small thickness direction retardation.

Further, in the optical film according to the present invention, a viewing angle, a contrast ratio, and color shift can be improved in an IPS mode display device due to a small phase difference.

### BEST MODE

The present invention relates to an optical film with good optical properties, and particularly, to an optical film with good optical properties in which a contrast ratio having an in-plane retardation of 0 to 10 nm and a thickness direction retardation of −10 to 10 nm, and color shift are improved.

More specifically, the present invention relates to an optical film in which Re (λ) and Rth (λ) are satisfied with the following Formulae (I) and (II).

0≦Re(587)≦10 (I)

−10≦Rth(587)≦10 (II)

(In the Formulae, Re (λ) represents an in-plane retardation (unit: nm) at a wavelength of λ nm, and Rth (λ) represents a film thickness direction retardation (unit: nm) at a wavelength of λ nm.)

It is preferable that the present invention contains, as an additive for satisfying these conditions, at least one compound represented by the following Chemical Formula 1.

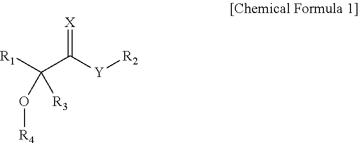

In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl,

R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl,

R₁, R₂, R₃ and R₄ are not hydrogen at the same time, and

alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ may be further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.

Each configuration of the present invention will be described in detail below.

First, with respect to the optical film, a density thereof is not limited, but is 1.2 to 1.35.

A base film for the optical film is not particularly limited as long as the film may be a polymer film which is commonly used. Examples of the base film for the optical film that may be used include cellulose acetate, polycarbonate, polymethyl methacrylate, polyester, polyimide, polystyrene or a copolymer film of these polymer materials. As for a more substantial example, the cellulose acetate film as the base film is preferable for an optical structure or a display device.

The cellulose acetate used for the base film for the optical film according to the present invention refers to an ester formed of cellulose and acetic acid, in which all or a part of the hydrogen atoms of respective hydroxyl groups at 2-, 3- and 6-positions of a glucose unit constituting the cellulose are substituted with an acetyl group. A substitution degree may be measured according to ASTM D-817-91. The substitution degree of the cellulose acetate is, but not limited to, preferably 2.7 or more, and more preferably 2.7 to 3.0 which is effective to reduce the retardation of the cellulose acetate film.

The molecular weight of the cellulose acetate used for the base film for the optical film of the present invention is, but not limited to, a range of 200,000 to 350,000 as a weight average molecular weight. Furthermore, the molecular weight distribution of the cellulose acetate Mw/Mn (Mw refers to a weight average molecular weight and Mn refers to a number average molecular weight) is preferably 1.4 to 1.8. A molecular weight distribution of 1.5 to 1.7 is preferable for realizing a transmittance and a mechanical physical property suitable for the optical film.

Next, other additives used for the optical film will be described.

To a solution (dope) to be used for a solvent casting method, a great variety of other additives, such as plasticizers, anti-ultraviolet agents, deterioration inhibitors, fine particles, peeling agents, infrared absorbing agents, optical anisotropy controlling agents, or the like, may be added according to use in respective preparation processes. Specific types of the additives are used without limitation as long as the additives are commonly used in the related art. It is preferable that the content of the additive to be used is in a range of not reducing physical properties of the optical film. The period of time when other additives are added is determined according to the type of the additives. An additive adding process may be carried out at the last of dope preparation.

The plasticizers are used to improve the mechanical strength of the film. When the plasticizers are used, a drying process time of the film may be reduced. The plasticizers are used without limitation as long as they are used commonly. Examples of the plasticizers include phosphoric acid ester and phthalic acid ester or carboxylic acid ester selected from citric acid ester, and the like. Examples of the phosphoric acid ester include triphenyl phosphate (TPP), biphenyl diphenyl phosphate, tricresyl phosphate (TCP), and the like. Examples of the phthalic acid ester include dimethyl phthalate (DMP), diethyl phthalate (DEP), dibutyl phthalate (DBP), dioctyl phthalate (DOP), diphenyl phthalate (DPP), diethyl hexyl phthalate (DEHP), and the like. Examples of the citric acid ester include o-acetyl triethyl citrate (OACTE), o-acetyl tributyl citrate (OACTB), and the like. Examples of other carboxylic acid ester include butyl oleate, methyl acetyl lysine oleate, dibutyl sebacate and a great variety of trimellitic acid ester. Preferably, phthalic acid ester (DMP, DEP, DBP, DOP, DPP, and DEHP) plasticizers may be used. The content of the plasticizer to be used is 2 to 20 parts by weight and more preferably 5 to 15 parts by weight, with respect to 100 parts by weight of the base resin for the optical film.

Examples of the anti-ultraviolet agents to be used include a hydroxybenzophenone-based compound, a benzotriazole-based compound, a salicylic acid ester-based compound, a cyanoacrylate-based compound, and the like. The amount of the anti-ultraviolet agents to be used is 0.1 to 3 parts by weight, preferably 0.5 to 2 parts by weight, with respect to 100 parts by weight of the base resin for the optical film.

Examples of the deterioration inhibitors which may be used include antioxidants, peroxide decomposers, radical inhibitors, metal inactivating agents, deoxygenating agents, optical stabilizers (hindered amine or the like). Examples of the preferable deterioration inhibitors include butylated hydroxytoluene (BHT), tribenzyl amine (TBA), and the like. The amount of the deterioration inhibitors to be used is preferably 0.01 to 5 parts by weight, more preferably 0.1 to 1 part(s) by weight, with respect to 100 parts by weight of the base resin for the optical film.

The fine particles are added in order that the film satisfactorily maintains curl inhibition, transportability, adhesion prevention in a roll shape or damage resistance, and may use any one selected from an inorganic compound or an organic compound. Preferable examples of the inorganic compound include a silicon-containing compound, silicon dioxide, titanium oxide, zinc oxide, aluminum oxide, barium oxide, zirconium oxide, strontium oxide, antimony oxide, tin oxide, tin oxide.antimony, calcium carbonate, talc, clay, calcined kaolin, calcined calcium silicate, calcium silicate hydrate, aluminum silicate, magnesium silicate, calcium phosphate, and the like. More preferably, a silicon-containing inorganic compound or zirconium oxide may be used. The fine particles have an average primary particle size of 80 nm or less, preferably 5 to 80 nm, more preferably 5 to 60 nm, and still more preferably 8 to 50 nm. When the average primary particle size of the fine particles is larger than 80 nm, the surface smoothness of the film is damaged.

Next, additives used for the present invention will be described in detail. The additives are added to reduce a thickness direction retardation (Rth) of the optical film and preferably compounds represented by the following Chemical Formula 1.

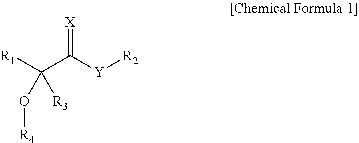

(In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl,

R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl,

R₁, R₂, R₃ and R₄ are not hydrogen at the same time, and

alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ may be further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.)

Substituents containing the terms “alkyl”, “alkoxy”, and another term “alkyl” moieties described herein contain a straight-chain or branched form, the term “alkenyl” includes a straight-chain or branched form which has at least one double bond and 2 to 8 carbon atoms.

The term “(C3-C20)cycloalkyl” described herein a saturated monocyclic ring structure form or saturated bicyclic ring structure form having 3 to 20 carbon atoms.

The term “aryl” described herein is an organic radical derived from aromatic hydrocarbon by removal of one hydrogen atom, and contains monocyclic or fused ring system including 4 to 7 ring atoms, preferably 5 to 6 ring atoms for each ring. Specific examples thereof include phenyl, naphthyl, biphenyl, tolyl, and the like, but are not limited thereto.

The term “heterocycloalkyl” described herein refers to a cycloalkyl group in which a saturated cyclic hydrocarbon skeleton atom is one to three hetero atom(s) selected from N, O, and S and the residual saturated monocyclic or bicyclic ring skeleton atom is carbon. Examples of the heterocycloalkyl include pyrrolidinyl, azetidinyl, pyrazolidinyl, oxazolidinyl, piperidinyl, piperazinyl, morpholinyl, thiomorpholinyl, thiazolidinyl, hydantoinyl, valerolactamyl, oxiranyl, oxetanyl, dioxolanyl, dioxanyl, oxathiolanyl, oxathianyl, dithianyl, dihydrofuranyl, tetrahydrofuranyl, dihydropyranyl, tetrahydropyranyl, tetrahydropyridinyl, tetrahydropyrimidinyl, tetrahydrothiophenyl, tetrahydrothiopyranyl, diazepanyl, azepanyl and the like.

The term “heteroaryl” described herein refers to an aryl group in which an aromatic ring skeleton atom is one to three hetero atom(s) selected from N, O, and S and the residual aromatic ring skeleton atom is carbon. The heteroaryl group includes a divalent aryl group in which a hetero atom in a ring is oxidized or quaternarized, for example, to form N-oxide or a quaternary salt. Specific examples thereof include furyl, thiophenyl, pyrrolyl, pyranyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, and the like, but are limited thereto.

More specifically, Chemical Formula 1 may be represented by the following Chemical Formula 2, 3 or 4.

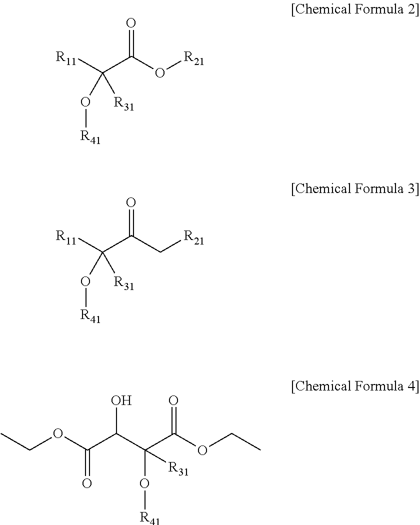

R₁₁ and R₂₁ are each independently selected from hydrogen, (C1-05)alkyl, (C6-C10)aryl, (C3-C20)cycloalkyl, (C3-C10)heterocycloalkyl, and (C4-C10) heteroaryl,

R₃₁ is selected from hydrogen, (C1-C5)alkyl, (C6-C10)aryl, and (C1-C10)alkoxy, R₄₁ is selected from hydrogen, (C1-C5)alkyl, carbonyl(C1-C5)alkyl, and (C6-C10)aryl, and

alkyl, aryl, cycloalkyl, alkoxy, carbonyl alkyl, heterocycloalkyl, and heteroaryl of Rn, R₂₁, R₃₁ and R₄₁ may be further substituted with at least one selected from (C1-C7)alkyl, halogen, nitro, cyano, hydroxy, and amino.

However, at least two of R₁₁, R₂₁ and R₃₁ in Chemical Formula 2 or 3 include a ring-type substituent selected from (C3-C20)cycloalkyl, (C6-C20)aryl, (C3-C20)heterocycloalkyl, and (C4-C20)heteroaryl.

More specifically, the compounds represented by Chemical Formula 1 include the compounds represented by the following Chemical Formula, but are not limited thereto.

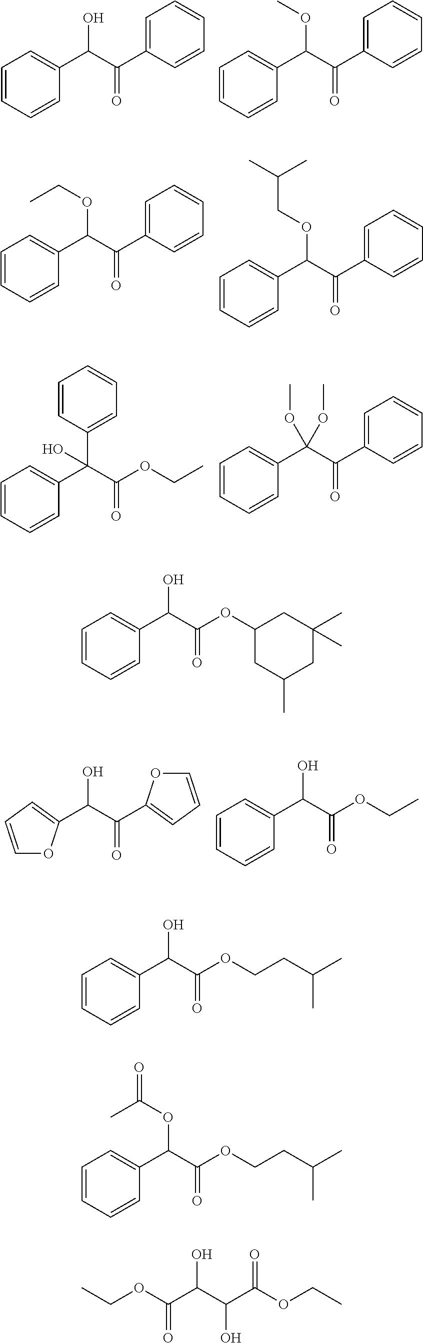

By including such additives, a good effect of reducing a thickness direction retardation may be exhibited, compared to addition of existing plasticizers, and optical properties such as stability and moisture resistance in high temperature and high humidity may be improved significantly.

As necessary, other optical anisotropy controlling agents, wavelength dispersion regulators, or the like may be further added, and other additives may be used without limitation as long as they are used commonly in the related art.

Next, a method of producing the optical film according the present invention will be described. The following optical film composition, that is, a dope solution is prepared in order to produce the optical film in the present invention.

It is preferable that the optical film composition contains, as an additive, 1 to 20 part(s) by weight of at least one selected from the following Chemical Formula 1 with respect to 100 parts by weight of the base resin.

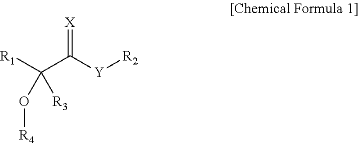

(In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl,

R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl, and R₁, R₂, R₃ and R₄ are not hydrogen at the same time, and

alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ may be further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.)

It is preferable that a solid concentration in the dope herein is 15 to 25 wt %, preferably 16 to 23 wt %. When the solid concentration in the dope is less than 15 wt %, it is difficult to form a homogeneous film due to too high fluidity. When the solid concentration in the dope is more than 25 wt %, the optical film composition is hardly dissolved and thus it is difficult to form a homogeneous film.

The content of the cellulose acetate used for the base resin for the optical film is 70 wt % or more, preferably 70 to 90 wt %, more preferably 80 to 85 wt %, based on the total content of the solid. Furthermore, with respect to cellulose acetate, at least two cellulose acetates of which substitution degrees, polymerization degrees or molecular distributions are different from each other may be mixed and used.

Furthermore, it is preferable that the cellulose acetate used for the base resin for the optical film is formed into a particle. At this time, it is preferable that 90 wt % of the cellulose acetate particles has an average particle size of 0.5 to 5 mm. Furthermore, it is effective that 50 wt % of the cellulose acetate particles has an average particle size of 1 to 4 mm.

It is preferable that the cellulose acetate particle has a shape similar to a sphere as possible. The cellulose acetate particles are dried so as to have a water content of 2 wt % or less, more preferably 1 wt % or less, and then to produce a dope solution, which is effective to exhibit physical properties when a film is formed.

It is preferable that the additives are used in a range of 1 to 20 part(s) by weight with respect to 100 parts by weight of the base resin for the optical film. When the additive is used in this range, a target phase difference range may be achieved. The additive less than 1 part by weight has a small effect of reducing a retardation. The additive more than 20 parts by weight may cause expense increase and a bleed out.

It is preferable that the optical film is formed by a solvent casting method in which a dope solution is used. In the solvent casting method, the composition of the optical film is dissolved in a solvent, the dissolved solution (dope) is cast on a support, and then a solvent is evaporated to form a film.

When a film is formed by this method, the solvent for preparing the composition (dope) for the optical film is preferably an organic solvent. Preferable examples of the organic solvent to be used include halogenated hydrocarbon. Examples of the halogenated hydrocarbon include chlorinated hydrocarbon, methylene chloride and chloroform. Of these, methylene chloride is most preferably used.

Furthermore, as necessary, an organic solvent other than the halogenated hydrocarbon is mixed and used. Examples of the organic solvent other than the halogenated hydrocarbon include ester, ketone, ether, alcohol, and hydrocarbon. Examples of the ester that may be used include methyl formate, ethyl formate, propyl formate, pentyl formate, methyl acetate, ethyl acetate, pentyl acetate, and the like. Examples of the ketone that may be used include acetone, methyl ethyl ketone, diethyl ketone, diisobutyl ketone, cyclopentanone, cyclohexanone, methylcyclohexanone. Examples of the ether that may be used include diisopropyl ether, dimethoxymethane, dimethoxyethane, 1,4-dioxane, 1,3-dioxolane, tetrahydrofuran, anisole, phenetole, and the like. Example of the alcohol that may be used include methanol, ethanol, 1-propanol, 2-propanol, 1-butanol, 2-butanol, t-butanol, 1-pentanol, 2-methyl-2-butanol, cyclohexanol, 2-fluoroethanol, 2,2,2-trifluoroethanol, 2,2,3,3-tetrafluoro-1-propanol, and the like.

More preferably, methylene chloride may be used as a main solvent, and alcohol may be used as a co-solvent. Specifically, methylene chloride and alcohol may be mixed with a weight ratio of 80:20 to 95:5, which is effective to homogeneously dissolve the base resin for the optical film in a solvent.

The optical film composition may be prepared by dissolution at normal temperature, high temperature or low temperature.

Preferably, the optical film composition has a viscosity of 1 to 400 Pa·s, more preferably 10 to 200 Pa·s, at 40° C.

The optical film may be prepared according to a common solvent casting method. More specifically, the prepared dope solution (the optical film composition) is stored first in a reservoir, and foams included in the dope solution are removed. The defoamed dope solution is supplied from a dope solution outlet to a press die by a press type metric gear pump capable of pumping a constant amount of fluid with high precision depending on a rotating speed. The dope solution is uniformly cast from a slit of the press die on a metal support which travels endlessly. At the peel point, where the metal support nearly completes a cycle, a still wet dope film (also called a web) is peeled off the metal support. Both ends of the prepared web are pinched with clips, and the web is transferred with a tenter while maintaining a width, and dried. Subsequently, the web is transferred to a roller of a dryer and dried, and wound by a given length with a winder.

During casting of the solution, a space temperature is preferably −50° C. to 50° C., more preferably −30° C. to 40° C., and most preferably −20° C. to 30° C. Since the optical film solution cast at low space temperature is instantaneously cooled on the support, thereby improving gel strength, a film in which a lot of organic solvent remains is formed. Accordingly, the film may be quickly peeled off the support without a need to evaporate the organic solvent from the optical film solution. Examples of the gas for cooling the space that may be used commonly include air, nitrogen, argon or helium. Preferably, relative humidity is 0 to 70%, most preferably 0 to 50%.

Preferably, the temperature of the support (casting portion) on which the optical film solution is cast is −50 to 130° C., more preferably −30° C. to 25° C., and most preferably −20 to 15° C. In order to cool the casting portion, a cooled gas may be introduced to the casting portion. Alternatively, a cooling device may be disposed at the casting portion to cool a space. During the cooling, it is important that water is not adhered to the casting portion. In a case where gas is used for the cooling, it is preferable that the gas is dried.

Also, the optical film may be surface-treated, if necessary. The surface treatment is carried out in general to improve adhesivity of the optical film. The surface treatment may include glow discharge treatment, UV treatment, corona treatment, flame treatment, saponification treatment, or the like.

The optical film may be stretched to control the degree of retardation. Preferably, the degree of stretching is −10 to 100%, more preferably −10 to 50%, most preferably −5 to 30%.

Preferably, the optical film has a thickness of 20 to 140 μm, more preferably 40 to 100 μm.

The optical film according to the present invention may be employed as optical structures such as an optical compensation lamination film, a hard coating film, an antiglare film, a low reflection film, an anti-reflection film, an optical filter for stereo-scopic image or a polarizing plate, and may be used as a single sheet or laminated into two or more sheets.

Furthermore, such an optical film or optical structure may be used to produce a liquid crystal display device.

Hereinafter, a detailed description of the present invention will be provided by way of an example. However, the present invention is not limited to the following Examples.

Hereinafter, physical properties of the film were measured as follows.

1) Optical Anisotropy

Re was measured using a birefringence analyzer (KOBRA-WPR, manufactured by Oji Scientific Instrument) by irradiating a film with light at a wavelength of 587 nm in a direction perpendicular to the film. Rth was measured by irradiating the film with light at a wavelength of 587 nm in a direction of 40 degrees from a perpendicular to the film toward the slow axis (determined by KOBRA-WPR) in the Re plane, determined using KOBRA-WPR.

2) Haze Measurement

Haze was measured with a Haze analyzer (Haze Meter HM-150, manufactured by Murakami Color Research Laboratory) according to ASTM D1003 Haze and Luminous Transmittance of Transparent Plastics standard.

3) Chromaticity Measurement

A chromaticity was expressed as a score by measuring an absorbancy of the film shown by absorption, reflection, and transmission from a light source with a colorimeter (ColorQuest XE, manufactured by Hunter Associates Laboratory) according to ASTM D1209 standard.

4) Moisture Resistance Measurement

A moisture resistance of the film was measured with a moisture permeability tester (PERMATRAN-W Model 3/33, manufactured by MOCON). Moisture which is permeated to an internal cell through the film from an external cell was measured under the condition of a pressure applied on a film specimen of 760 mmHg, a temperature of 37.8° C., a relative humidity (RH) of the external cell of 100%, and N2 carrier gas.

5) High Temperature and High Humidity Stability

A film specimen was left for 150 hours under the condition of relative humidity (RH) of 90% and a temperature of 60° C. in a thermo-hygrostat (KCL-2000, manufactured by EYELA), followed by annealing at a room temperature, and then thickness direction retardation (Rth), Haze, chromaticity were measured by the method described above, which was compared to an initial value.

### Example 1

Preparation of Optical Film Composition (Dope)

The compositions described in the following Table 1 were added to a stirrer and then dissolved in a temperature of 30° C.

2-(2H-Benzotriazol-2-yl)-6-(1-methyl-1-phenylethyl)-4-(1,1,3,3-tetramethylbutyl)phenol was used as the anti-ultraviolet agent of the following composition.

TABLE 1

| | Content |
|---|---|
| | (parts by |
| Component | weight) |
| Cellulose acetate powder having a | 100 parts by weight |
| substitution degree of 2.87 | |
| Additive (2-Methoxy-2-phenylacetophenone) | 10 parts by weight |
| Anti-ultraviolet agent | 2 parts by weight |
| Silicon dioxide, average particle size 16 nm | 0.5 parts by weight |
| Methylene chloride | 440 parts by weight |
| Methanol | 50 parts by weight |

The dope thus obtained was warmed to 30° C., pumped to a gear pump, filtered with a filter paper having an absolute filtration accuracy of 0.01 mm, and then filtered with a cartridge filter having an absolute filtration accuracy of 5 μm.

Preparation of Optical Film

The dope solution obtained by the filtration process was cast on a slanted stainless steel support using a casting die, and then peeled off. The content of the remaining solvent was controlled to be 25 wt % at the time of peeling. After connecting to a tenter, the film was stretched by 105% (% represents a length percent (%)) in the width direction. After the film exited from the tenter, both end portions at left and right sides of the film were cut by 150 mm. Then, the film was dried using a dryer. After the film exited from the dryer, both end portions of the film were cut by 30 mm. Then, knurling processing was performed at a width of 10 mm and a height of 68 μm from the end portion, and the film was wound in a roll shape. The film thus obtained has a thickness of 60 μm and a thickness direction retardation (Rth) of the optical film was measured by the method described above.

### Examples 2 to 8

Preparation of Optical Film

An optical film was produced in the same manner as Example 1, except that 2-Methoxy-2-phenylacetophenone in the compositions of Example 1 was changed to each type of additives described in the following Table 2.

TABLE 2

| | | Content |
|---|---|---|
| | | (parts by |
| | Type of Additive | weight) |
| Example 1 | 2-Methoxy-2-phenylacetophenone | 10 |
| Example 2 | 2-Ethoxy-2-phenylacetophenone | 10 |
| Example 3 | 2-Isobutoxy-2-phenylacetophenone | 10 |
| Example 4 | Ethyl 2-hydroxy-2,2- | 10 |
| | diphenylacetate | |
| Example 5 | 3,3,5-trimethylcyclohexyl 2- | 10 |
| | hydroxy-2-phenylacetate | |
| Example 6 | Ethyl 2-hydroxy-2,2- | 6.7 |
| | diphenylacetate | |
| | 3,3,5-trimethylcyclohexyl 2- | 3.3 |
| | hydroxy-2-phenylacetate | |
| Example 7 | Ethyl 2-hydroxy-2,2- | 5 |
| | diphenylacetate | |
| | 3,3,5-trimethylcyclohexyl 2- | 5 |
| | hydroxy-2-phenylacetate | |
| Example 8 | Ethyl 2-hydroxy-2,2- | 3.3 |
| | diphenylacetate | |
| | 3,3,5-trimethylcyclohexyl 2- | 6.7 |
| | hydroxy-2-phenylacetate | |

### Comparative Example 1

### Preparation of Optical Film Composition (Dope)

The following compositions were added to a stirrer and then dissolved in a temperature of 30° C.

In each composition of the following [Table 3], 2,2′-(ethane-1,2-diylbis(oxy))bis(ethane-2,1-diyl)dipentanoate manufactured by Fujifilm Corporation (U.S. Pat. No. 7,671,947) was used as the additive, and 2-(2H-Benzotriazol-2-yl)-6-(1-methyl-1-phenylethyl)-4-(1,1,3,3-tetramethyl butyl)phenol was used as the anti-ultraviolet agent.

TABLE 3

| | Content |
|---|---|
| | (parts by |
| Component | weight) |
| Cellulose acetate powder having a | 100 parts by weight |
| substitution degree of 2.87 | |
| Additive | 10 parts by weight |
| Anti-ultraviolet agent | 2 parts by weight |
| Silicon dioxide, average particle size 16 nm | 0.5 parts by weight |
| Methylene chloride | 440 parts by weight |
| Methanol | 50 parts by weight |

The dope solution thus obtained was warmed to 30° C., pumped to a gear pump, filtered with a filter paper having an absolute filtration accuracy of 0.01 mm, and then filtered with a cartridge filter having an absolute filtration accuracy of 5 μm.

Preparation of Optical Film

The dope solution obtained by the filtration process was cast on a slanted stainless steel support using a casting die, and then peeled off. The content of the remaining solvent was controlled to be 25 wt % at the time of peeling. After connecting to a tenter, the film was stretched by 105% (% represents a length percent (%)) in the width direction. After the film exited from the tenter, both end portions at left and right sides of the film were cut by 150 mm. Then, the film was dried using a dryer. After the film exited from the dryer, both end portions of the film were cut by 30 mm. Then, knurling processing was performed at a width of 10 mm and a height of 68 μm from the end portion, and the film was wound in a roll shape. The film thus obtained has a thickness of 60 μm, in which a thickness direction retardation (Rth) of the optical film was measured by the method described above.

A film was produced in the same manner as Comparative Example except that the prepared dope was used, and a result was shown in the following Table 4.

TABLE 4

| | | | | | | Stability at high | | |
|---|---|---|---|---|---|---|---|---|
| | | | | Moisture | | temperature and | | |
| | | | | Resistance | | high humidity | | |
| | | | Chromaticity | (g · μm/ | Haze | ΔRth | ΔHaze | |
| | Re (nm) | Rth (nm) | (b*) | m² · day) | (%) | (nm) | (% p) | Δb* |
| Comparative | 1.00 | 2.37 | 0.08 | 52214.80 | 0.47 | 3.13 | 0.34 | 0.07 |
| Example 1 | | | | | | | | |
| Example 1 | 0.47 | 6.07 | 0.24 | 45483.74 | 0.23 | 2.41 | 0.16 | 0.06 |
| Example 2 | 0.16 | 4.98 | 0.16 | 44091.37 | 0.21 | 2.10 | 0.12 | 0.05 |
| Example 3 | 0.21 | 6.98 | 0.11 | 43850.47 | 0.20 | 2.05 | 0.14 | 0.05 |
| Example 4 | 0.33 | 5.17 | 0.13 | 47341.83 | 0.16 | 0.40 | 0.25 | −0.02 |
| Example 5 | 0.47 | 4.37 | 0.12 | 44097.53 | 0.17 | 0.69 | 0.17 | −0.01 |
| Example 6 | 0.04 | 6.37 | 0.14 | 44656.12 | 0.16 | 0.44 | 0.21 | 0.01 |
| Example 7 | 0.11 | 5.97 | 0.13 | 46226.57 | 0.16 | 0.46 | 0.17 | −0.01 |
| Example 8 | 0.26 | 6.63 | 0.13 | 45463.70 | 0.15 | 0.55 | 0.18 | −0.01 |

As shown in Table 4, it could be confirmed that the optical film has a small Rth at a wavelength of 587 nm, a good stability at high temperature and high humidity and, a higher Haze and a higher moisture resistance, when the additive described in each Example is added unlike existing plasticizers.

1. An optical film, comprising:
at least one selected from compounds represented by the following Chemical Formula 1, as an additive. 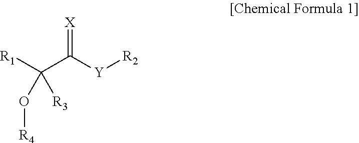
(In Chemical Formula 1, X represents O, S or N—R₅, Y represents O, S, N—R₂₅ or a chemical bond, R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl,
R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, and (C4-C20) heteroaryl, and R₁, R₂, R₃ and R₄ are not hydrogen at the same time, and
alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ each are further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl and (C4-C20)heteroaryl.)

2. The optical film of claim 1, wherein the compound represented by Chemical Formula 1 is selected from the following Chemical Formula 2, 3 or 4. 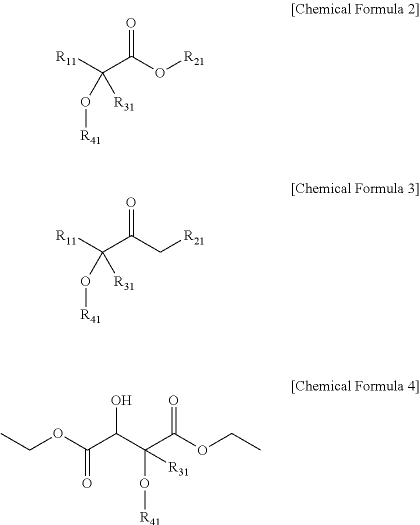
(In the Chemical Formulae, Rui and R₂₁ are each independently selected from hydrogen, (C1-C5)alkyl, (C6-C10)aryl, (C3-C20)cycloalkyl, (C3-C10)heterocycloalkyl, and (C4-C10) heteroaryl, R₃₁ is selected from hydrogen, (C1-C5)alkyl, (C6-C10)aryl, and (C1-C10)alkoxy, R₄₁ is selected from hydrogen, (C1-C5)alkyl, carbonyl(C1-C5)alkyl, and (C6-C10)aryl, and
alkyl, aryl, cycloalkyl, alkoxy, carbonyl alkyl, heterocycloalkyl, and heteroaryl of R₁₁, R₂₁, R₃₁ and R₄₁ each are further substituted with at least one selected from (C1-C7)alkyl, halogen, nitro, cyano, hydroxy, and amino.)

3. The optical film of claim 2, wherein Rut, R₂₁ and R₃₁ in Chemical formula 2 or 3, are each independently selected from hydrogen, (C1-C5)alkyl, (C3-C20)cycloalkyl, (C3-C10)heterocycloalkyl, (C6-C10)aryl, and (C4-C10) heteroaryl,
alkyl, cycloalkyl, heterocycloalkyl, aryl, and heteroaryl of R₁₁, R₂₁, and R₃₁ are further substituted with at least one selected from (C1-C7)alkyl, halogen, nitro, cyano, hydroxy, and amino,
provided that at least two of R₁₁, R₂₁ and R₃₁ include a ring-type substituent selected from (C3-C20)cycloalkyl, (C6-C20)aryl, (C3-C20)heterocycloalkyl, and (C4-C20)heteroaryl.

4. The optical film of claim 2, wherein the compound represented by Chemical Formula 2 or 3 is selected from the following Chemical Formula.

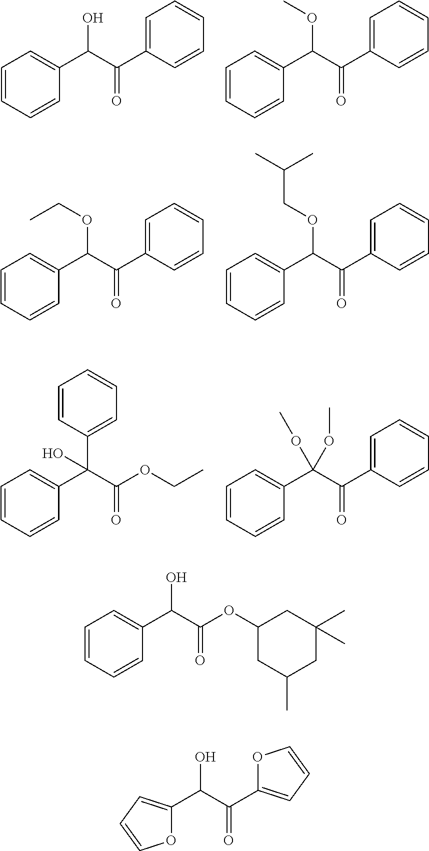

5. The optical film of claim 2, wherein the compound represented by Chemical Formula 2, 3 or 4 is selected from the following Chemical Formula.

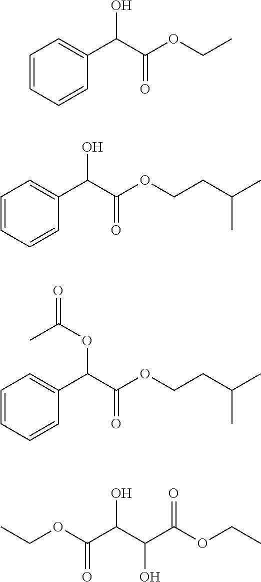

6. The optical film of claim 1, wherein Re (λ) and Rth (λ) are satisfied with the following Formulae (I) and (II).
0≦Re(587)≦10
−10≦Rth(587)≦10
(In the Formulae, Re (λ) represents an in-plane retardation (unit: nm) at a wavelength of λ nm, and Rth (λ) represents a film thickness direction retardation (unit: nm) at a wavelength of λ nm.

7. The optical film of claim 1, wherein the optical film is a cellulose acetate film having a substitution degree measured according to ASTM D-817-91 of 2.7 to 3.0.

8. An optical film composition, comprising:
1 to 20 part(s) by weight of at least one selected from the compounds in the following Chemical Formula 1, as an additive, with respect to 100 parts by weight of a base resin. 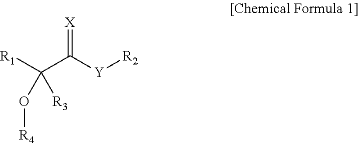
(In Chemical Formula 1, X represents O, S or N—R₅, Y represents O, S, N—R₂₅ or a chemical bond,
R₁, R₃, R₅ and R₂₅ are each independently selected from hydrogen, (C1-C10)alkyl, (C6-C20)aryl, (C1-C20)alkoxy, (C3-C20)cycloalkyl, (C2-C7)alkenyl, (C1-C10)alkoxycarbonyl(C1-C10)alkyl, carbonyl(C1-C10)alkyl, (C3-C20)heterocycloalkyl, and (C4-C20)heteroaryl,
R₂ and R₄ are each independently selected from hydrogen, (C1-C10)alkyl, (C6-C20)aryl, (C3-C20)cycloalkyl, carbonyl(C1-C10)alkyl, (C3-C20)heterocycloalkyl, (C4-C20) heteroaryl,
R₁, R₂, R₃ and R₄ are not hydrogen at the same time, and
alkyl, aryl, alkoxy, cycloalkyl, alkenyl, alkoxycarbonyl alkyl, carbonyl alkyl, heterocycloalkyl, and heteroaryl of R₁, R₂, R₃, R₄, R₅ and R₂₅ each are further substituted with at least one selected from (C1-C7)alkyl, halogen, nitro, cyano, hydroxy, amino, (C6-C20)aryl, (C2-C7)alkenyl, (C3-C20)cycloalkyl, (C3-C20)heterocycloalkyl or (C4-C20)heteroaryl.)

9. The optical film composition of claim 8, further comprising, as an additive, at least one or two selected from an anti-ultraviolet agent, a fine particle, a plasticizer, a deterioration inhibitor, a peeling agent, an infrared absorbing agent, and an optical anisotropy controlling agent.

10. An optical structure using the optical film of claim 1.

11. The optical structure of claim 10, wherein the optical structure is an optical compensation lamination film, a hard coating film, an antiglare film, a low reflection film, an anti-reflection film, an optical filter for stereo-scopic image or a polarizing plate.

12. A display device manufactured by using the optical film of claim 1.

13. A display device manufactured by using the optical structure of claim 10.
